SEQUENCE LISTING (2 sequences)

SEQ ID NO: 1 - DNA, 21 bp, Homo sapiens
  source 1..21: mol_type = genomic DNA
ggaggagaccccattctatac

SEQ ID NO: 2 - DNA, 23 bp, Homo sapiens
  source 1..23: mol_type = genomic DNA
gctcagaccatacctatgtatcc

(19) United States
(12) Patent Application Publication
(10) Pub. No.: US 2025/0170575 A1
(43) Pub. Date: May 29, 2025

(54) DRY SWAB COLLECTION SYSTEM AND METHOD

(71) Applicant: MATERIALS AND MACHINES CORPORATION OF AMERICA, Lincoln, NE (US)

(72) Inventors: Abraham Oommen, Lincoln, NE (US); Yashitola Wamboldt, Eagle, NE (US)

(21) Appl. No.: 18/882,467

(22) Filed: Sep. 11, 2024

Related U.S. Application Data
(60) Provisional application No. 63/537,765, filed on Sep. 11, 2023.

(51) Int. Cl. B01L 3/00

(52) U.S. Cl. CPC B01L 3/505; B01L 2200/0689; B01L 2200/185; B01L 2300/042; B01L 2300/0832; B01L 2300/123

(57) ABSTRACT

A system and method of collection of a biological specimen includes obtaining the biological sample using a swab through conventional techniques (e.g. a cheek swab may be obtained according to the instructions in the instructions section), inserting the swab into the flexible collection tube and sealing the tube, where the method and system may further include inserting the flexible collection tube containing the swab into a vacuum bag. The method and system may further include vacuum sealing the vacuum bag to remove air and moisture from the vacuum bag and flexible collection tube to stabilize the biological sample for long term storage.

This application claims the benefit of U.S. Provisional Application No. 63/537,765 entitled “DRY SWAB COLLECTION SYSTEM AND METHOD” filed Sep. 11, 2023, which is incorporated by reference in its entirety. This application includes the material in the Sequence Listing XML file, entitled M1864-00162US created on Feb. 18, 2025, having a size of 3 KB, which is incorporated by reference in its entirety.

### BACKGROUND

Sample collection for diagnostic or storage purposes is a common and accepted method throughout the world. In most cases, various types of swabs are used and different types of swabs for sample collection have been around since the early 1920s. They have evolved from cotton applicators to totally synthetic flocked swabs used today, for a variety of applications. Different types of swabs are used for different sample types also. Additionally, where the sample is collected from (part of the body or from a specific location like a drain) and from what species and or what purpose also determines the type of swab being used. Specimen collection swabs are used to collect specimens for further testing. Depending on the nature of the test, the specimen collected maybe analyzed for DNA, RNA, proteins or other inorganic materials as well. For example, a discussion of conventional specimen collection swabs and techniques is discussed by Puritan Medical Products (see, A History of Specimen Collection: Swabs Then and Now, Puritan Blog, May 1, 2019, referenced in the Information Disclosure Statement).

Typically, most swabs are designed, manufactured and used in well known and standard ways. For example, with the Covid-19 pandemic, the use of flocked swabs have gone up globally. The public, though familiar with swabs may not have been acquainted with them intimately until the global demand of testing increased due to the pandemic. Generally speaking, a swab can be represented as being a piece of thin plastic or wood that is approximately 6 inches (15 cm) long with a proximal and a distal end. This is shown in FIG. 1 which represents a commonly used plastic swab with break point. The proximal end is slightly thicker (mostly in the case of plastic swabs) and is used to hold the swab. The proximal end is indicated as 104. The distal end is slightly thinner and ends in a conical shape with about an inch or so of fibrous material (nylon, cotton or other natural or synthetic material) making up the tip of the swab. This is indicated as 101 in FIG. 1. This tip is where the specimen is technically “collected”. Also, somewhere in the middle or about 3.25 inches (82 to 85 cm) from the tip, there is a break point. This break point is seen mostly in plastic swabs and is indicated as 103. The part between the break point and the tip is indicated as 102.

Conventional wooden swabs are mostly cotton tipped swabs and are typically one-time use swabs and do not have a break point. They are used as specimen collection swabs for moving sample into a culture media or to a rapid test device (e.g. like a Strep test seen in a clinic). The focus of this patent application will be with the commonly used plastic swab, which is used conventionally to collect a sample for short term storage in a collection media, and shipping to a lab for analysis or for long-term storage of the sample. For this, the collected specimen, in other words, the swab with the specimen on the tip 101 as indicated in FIG. 1, is deposited into a collection tube. Typically, this collection tube is narrow and long and has a medium for transport, conventionally referred to as “transport media”, “storage media” or a “collection media.” The liquid media is used as the transport, storage or collection media. This point is emphasized, for example, by the sale of sample collection tube and swabs with such transport, storage, or collection media (e.g., see Puritan PurSafe® 1 ml Molecular Preservative and 6″ Sterile Large Flock Swab (VL 1201 SAFE-H) and Puritan PurSafe® 1 ml Moelcuarl Preservative (VL 1201 SAFE), referenced in Information Disclosure Statement). The conventional purpose of the conventional swab is to collect specimens and deposit the specimen to a collection tube having a media for collection, transport, and storage.

Discussion of conventional swab collection techniques emphasize that samples are collected into a protective collection or transport media. Additionally, conventional DNA collection kits utilize a protective collection or transport media (e.g. Circle DNA, Family Tree DNA, 23 and Me, Ancestry, and BasePaws).

Whatever the testing situation or kit used may be, the liquid media in the collection tube varies depending on the sample and there is enough of it to cover the tip of the conventional swab. These media are typically buffers that keep the collected sample viable until the sample is used for further testing or analysis. This concept and practice is familiar to anyone trained in this art and hence its universal acceptance as a standard procedure. Once the conventional swab with the specimen is placed in the collection tube, the conventional swab is typically broken at the break point so that the swab itself is left in the collection tube. In other words, the collection tube is long enough to hold the broken swab (at the break point). This allows safe collection of hazardous materials (bacteria or virus) by keeping the collected sample inside the collection tube and not removing it to the outside. The broken off, distal part, that is the handle part of the conventional swab, is typically discarded.

As an example of a conventional collection swab, collection tube and method, the collection tube used is the standard collection tube found in most rapid at home antigen test kits. These are commonly referred to in the industry as “sample extraction tubes” or “antigen reagent tubes” and they are made mostly of low-density polyethylene (LDPE). This makes the tubes flexible so that fingers can be used to press the tube down with the swab inside it to squeeze the swab or to remove liquid from the tube by exerting pressure on the tube. The main body of the tube is marked as 204 in FIG. 2. The part where the swab tip ends up is marked as 208 in all figures including FIGS. 2, 3 and 4. Squeezing of the tube when the swab is inside (to release sample collected by the swab) is a standard procedure found in any or all rapid antigen testing kits. In conventional uses, the extraction tube has some type of a buffer in it (mostly in area marked 208) where the collected specimen (for example a nasal swab) is placed and then mixed well by squeezing the tube. The liquid inside the tube is indicated as 209 in FIG. 2. The same tube is then used to squeeze the sample out, for example, to a lateral flow device which has a specific test reagent inside it. Such tubes have a cap, marked as 205 in FIG. 2. In conventional uses, as shown in FIG. 3, the cap can be removed from the threaded part of the tube by twisting it. Typically, the cap 205 is removed as shown in FIG. 3 to place the swab with specimen inside the tube. Once the swab tip enters the liquid collection, storage, or transport media or buffer inside the tube (indicated as 209 in all figures), the swab is twisted and broken off at the break point (103). The cap is then placed back on the tube and twisted shut as shown in FIG. 5. In FIGS. 3 and 4, a smaller cap is also shown (marked as 206) which is attached to the larger cap. The smaller cap can also be removed by twisting the cap open as shown in FIG. 4. In these conventional uses, samples inside can be moved to, for example, a lateral flow device after removing the small cap 206. A detailed outline of the use of such a tube in a conventional antigen testing scenario is shown in FIG. 11. This example shows how such a tube will be used for Covid-19 testing in a commonly used antigen test format.

It is therefore desirable to have a swab collection system and method that does not utilize liquid media or any other buffer or storage media, and allows for long term storage of the swab and therefore the sample for at least thirty days, and more preferably one year or more.

### SUMMARY

In aspects, a biological specimen collection system for long term storage including a swab for collecting the biological specimen, a flexible collection tube, and a sealed vacuum bag, the sealed vacuum bag including a vacuum bag that is vacuum sealed to remove air from the vacuum bag, and flexible collection tube, and where the system does not comprise any media for collection, transport, or storage of the biological specimen.

The system of paragraph [0008], wherein the flexible collection tube comprises a main body made of a low-density polyethylene material.

The system of paragraph [0008], wherein the flexible collection tube further comprises a cap that is in removable attachment to the main body, and a smaller cap in removable screw attachment to the cap.

In aspects, a biological specimen collection system for storage of a biological sample including a swab for collecting the biological specimen, and a flexible collection tube, and where the system does not comprise any media for collection, transport, or storage of the biological specimen.

In aspects, the system of paragraph [0011], wherein the flexible collection tube comprises a main body made of a low-density polyethylene material.

In aspects, the system of paragraph [0011], wherein the flexible collection tube further comprises a cap that is in removable attachment to the main body, and a smaller cap in removable screw attachment to the cap.

In aspects, a method for collection and storage of a biological specimen without the use of any media for collection, transport, or storage of the biological specimen, the method including collecting the biological specimen using a swab, inserting the swab having the collected biological specimen into a flexible collection tube, and inserting the flexible collection tube into a vacuum bag to stabilize the biological sample, and storing the stabilized biological sample for at least 14 days.

The method of paragraph [0014], wherein the flexible collection tube includes a cap that is in removable attachment to the main body, and a smaller cap in removable screw attachment to the cap.

The method of paragraph [0015] further including after the insertion of the flexible collection tube into the vacuum bag, vacuum sealing the vacuum bag to remove air from the flexible collection tube and vacuum bag to stabilize the biological sample, wherein the small cap of the flexible collection tube is partially unscrewed, and wherein the storing of the stabilized biological sample for long term storage of at least 30 days.

### DESCRIPTION OF THE FIGURES

FIG. 1 represents a conventional plastic swab having a break point.

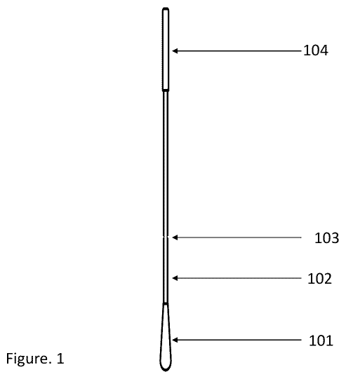

FIG. 2 represents a conventional collection tube having a liquid transport media.

FIG. 3 represents the conventional collection tube receiving a conventional swab.

FIG. 4 represents the conventional collection tube with a small cap removed to dispense a sample contacted with the liquid transport media.

FIG. 5 represents the conventional collection tube with the small cap attached to the conventional collection tube.

FIG. 6 represents a flexible collection tube having a swab with a collected biological sample.

FIG. 7 represents the flexible collection tube having the swab with the collected biological sample in a vacuum bag for long-term storage.

FIG. 8 represents the results of real-time PCR gel electrophoresis on collected biological samples using the system and method.

FIG. 9 represents the results of real-time PCR on collected biological samples using the system and method.

FIG. 10 represents the results of real-time PCR on a collected biological sample after long-term storage using the system and method.

FIG. 11 represents a conventional use of a conventional collection tube for COVID-19 Antigen testing.

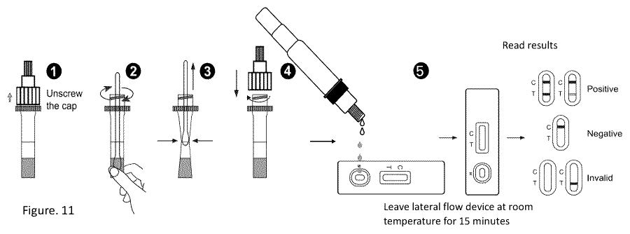

FIG. 12 represents a method of long term storage of a biological sample using the system and method.

FIG. 13 represents a method of sample collection.

### DETAILED DESCRIPTION OF THE INVENTION

A system and method of biological sample collection and long term storage using flexible sample tubes, swabs, and vacuum bags for shipping and long term storage of biological samples at room temperature conditions for molecular analysis is described. Swabs of various types are used to collect nasal, oral, vaginal or anal specimens for the detection of DNA or RNA from the collected source or from a resident infectious or non-infectious agent present on or inside the collected source. Samples may also be collected from a living (e.g. human) or non living source (e.g. a drain or pond) (a biological sample). The collected biological sample may contain a living organism, DNA, RNA or protein or even a chemical agent (analyte), that is subject to analysis with the purpose of detecting the presence or absence of the analyte) from the collected biological sample. The present method relates to using a swab to collect a biological sample without the use or presence of a collection, storage or transport media that is in liquid form and found in the collection tube.

### Media Free Collection of Specimen Examples

### Swab Use Instruction

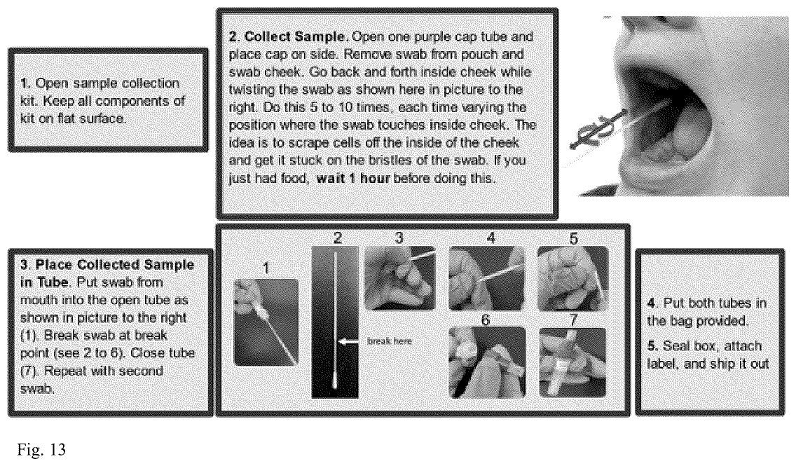

Flocked swabs purchased from vendors like Puritan (USA) and or CleanMo (China) were used to evaluate media free collection using the system and method. Volunteers were given swabs and told to swab the inside of their cheeks by following specific instructions. A general description of the instructions provided to volunteers are summarized in FIG. 13. The method may be used with any type of instruction may be provided that will result in the collection of cheek cells and they will vary slightly in its emphasis and mode but results in the same final outcome, that is cheeks cells are collected. In summary, sample collection involves using a swab, swabbing the inside of the mouth (above the gum line) but on the inside of the cheek area rather than the gum. This is to ensure that cheek cells are collected efficiently and easily. The swab is then placed in a flexible collection tube 200 that does not have any liquid of any type inside it.

### Collection Tube

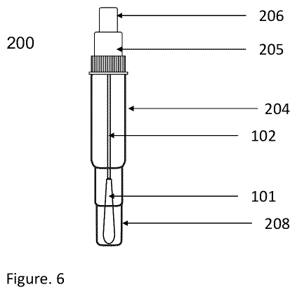

In the system and method, a flexible collection tube 200 is used. The flexible sample collection tube is shown in FIG. 6. The flexible collection tube 200 is a conventional sample collection tube of LDPE without any buffer or transport media.

Use of Antigen Extraction Tube in Current System and Method: In the present system and method, the flexible collection tube 200 serves three purposes:
1. As a swab holder after the specimen is collected on the swab.
2. As a swab holder after the specimen is collected on the swab as in 1 above, but without a sample collection, transport or storage media or buffer that is in liquid form. In other words, the tube is devoid of any liquid whatsoever.
3. As a swab holder as described in 1 and 2 above but with the purpose of evacuating the tube of any air post placement of the specimen containing swab in the tube and the use of the tube as a storage container for the collected specimen.

The purposes as described in 1 and 2 above is shown in FIG. 6. The specialized collection flexible tube is shown with a biological sample (collected specimen) inside it. This time, the liquid part (marked 209 in other figures) is deliberately shown as missing or absent. This is because in the system and method it was determined that liquid of any type, meaning, a buffer, sample collection, storage or transport media is not needed to collect specimen as long as the intent is to detect nucleic acids from the collected sample.

Flexible Tube for Sample Collection without Liquid Media or Buffers

### Collected Samples Can be Shipped Safely

In examples to demonstrate efficacy of the system and method, numerous samples were collected by volunteers in the specialized collection tube using swabs provided and above instructions provided to them. After collection in tubes without any liquid (dry sample collection), the samples were mailed back using a standard mailer provided for the purpose. Each volunteer was instructed to collect 2 samples from their cheeks using the two swabs provided and each swab was placed in a separate flexible collection tube 200. It took 5 to 14 days for the postal service to deliver the sample. One swab from each pair was tested to see if DNA was present as described below in the “Collected Samples have Functional DNA” section.

Collected Samples have Functional DNA

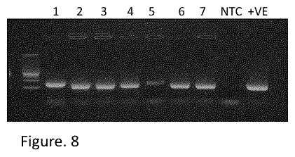

Dry swabs from 7 individuals who had collected samples in the flexible tubes were taken one at a time and DNA was extracted from it following standard DNA extraction procedures. A portion of the extracted DNA was used to perform standard PCR reaction using primers specific to the human Cox-1 gene. The PCR amplification of the DNA was done in a PCR machine (Solas 8, MatMaCorp, Lincoln, Nebraska), which is a thermal cycler and also a real-time PCR machine. The idea behind the experiment here is to show that human DNA was present in the sample even though no liquid media or buffer ever came in contact with the sample immediately after collection, shown in FIG. 8.

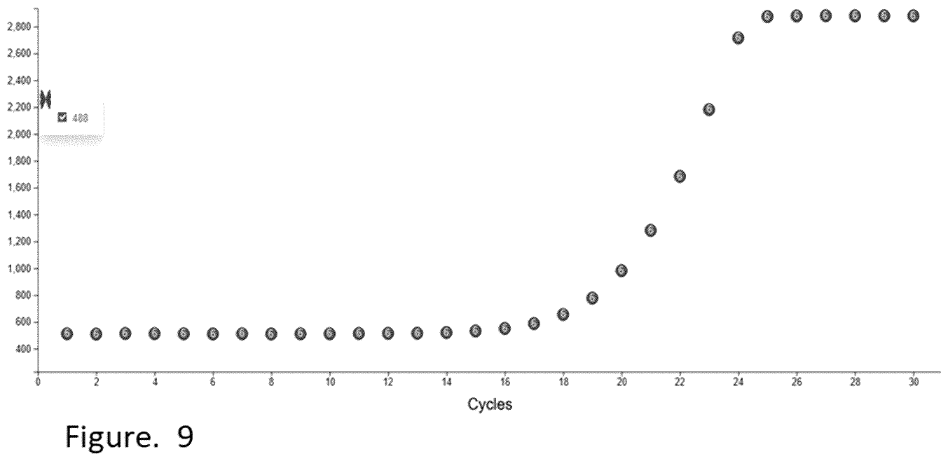

The expected 155 bp fragment that corresponds to the Cox-1 amplicon is shown from individuals identified as 1 to 7 above each lane. The PCR primers used were Human cox1-F (Forward Primer) with the sequence identified as SEQ ID No: 1 (5′-GGAGGAGACCCCATTCTATAC-3′) and the Human cox1-R (Reverse Primer) with the sequence identified as SEQ ID NO: 2 5′-GCTCAGACCATACCTATGTATCC-3′. The lane marked NTC is the negative control and does not show the expected band. The lane marked+VE is a positive control of previously purified human DNA and shows the expected size from the Cox-1 amplicon. The amplification was also monitored in real-time using EvaGreen dye (Biotium, San Francisco, California, USA) which was detected in the 520 nm Channel of the Solas 8 (MatMaCorp, Lincoln, Nebraska) device. A representative data from one of the samples is shown in FIG. 9, demonstrating that real-time PCR detection also works from samples collected dry.

### Flexible Extraction Tube for Sample Storage and Banking

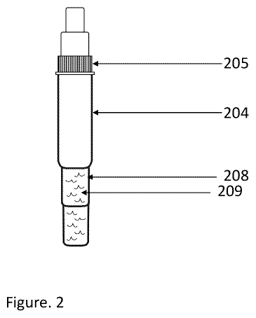

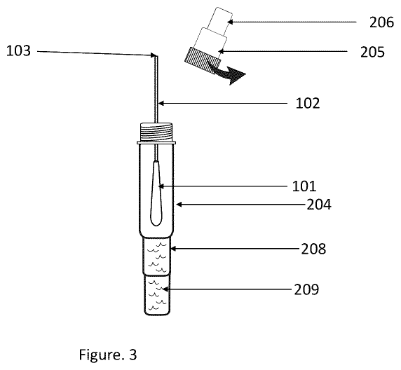

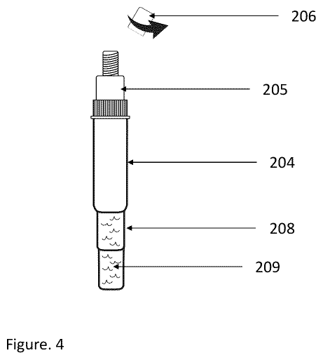

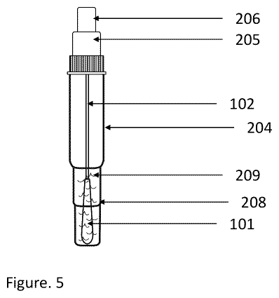

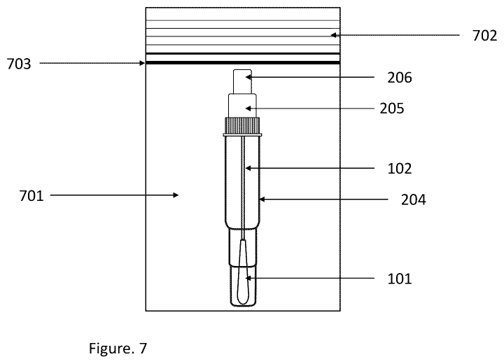

FIG. 6 represents the flexible collection tube 200 having a collected biological sample, and FIG. 7 shows the system (flexible collection tube 200 and vacuum bag) having a collected biological sample. The flexible collection tube 200 has a flexible body made of low-density polyethylene (LDPE). This feature along with small cap (206 in FIG. 6) over the large cap (205 in FIG. 6) (where 205 and 205 are the same as described in FIGS. 2-5) allows a collected sample as depicted in FIG. 7 to be stored in a specialized plastic bag (marked as 701 in FIG. 7) for the purpose of long-term storage. Long term storage of a biological specimen, for example a cheek swab from a human, is the example shown here. Any cheek swab collected as shown in FIG. 6 can be placed in a specialized bag, marked 701 as shown in FIG. 7. The top of the bag 701 sometimes comes with pressure sealable or zip-lock like sealable ends as depicted as 702 in FIG. 7. Most of the time, such sealable areas are lacking and instead a heat sealer within the vacuum sealer is able to create a heat seal indicated as 703 in FIG. 7. The specialized bag is a plastic bag made of nylon and polyethylene. These types of bags are commonly used for long term storage of food using a vacuum sealer and are commonly referred to as vacuum sealer bags. The flexible collection tube 200 with collected biological sample in a vacuum sealer bag, is then placed in a vacuum sealer to remove air from the bag as well as from the flexible collection tube 200 having the collected biological sample. To facilitate air removal from the flexible collection tube 200 with sample, cap 206 is slightly twisted loose on cap 205 before being placed in a vacuum bag as shown in FIG. 7. After the flexible collection tube 200 is placed in the vacuum bag 701, the vacuum bag 701 is placed in a standard vacuum sealer with a heat sealer and instructions from the manufacturer of the sealer are followed to remove the air and vacuum seal the vacuum bag 701 with the flexible collection tube 200. Appropriate labels indicating the identity of the sample in the flexible tube are placed on the vacuum bag and the evacuated vacuum bag is stored away at room temperature.

The removal of air from the flexible tube and also from the vacuum bag remove (reduces) moisture (humidity) from the collected biological sample as well as from the flexible tube and vacuum bag. Removal of moisture from cells collected from a host or environment prevents (reduces, delays) mold, bacteria or other spoilage organisms or their spores from germinating and growing.

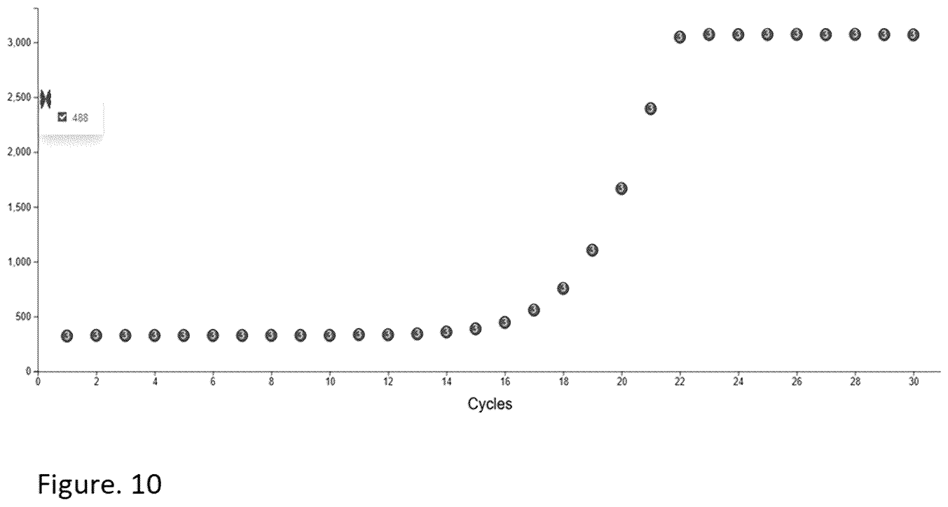

The drier the environment, the longer the survivability and ability of the sample to stay intact. Samples stored in this fashion at room temperature were randomly tested to see if DNA could be isolated. An example of a sample stored at room temperature for a month (long-term storage) after the air was removed using a vacuum sealer as described above is shown in FIG. 10.

DNA was extracted from stored samples following a standard protocols developed within the company and as described in the earlier section above. A portion of the extracted DNA was used to perform standard real-time PCR reactions using primers specific to the human Cox-1 gene. The PCR amplification of the DNA was done in a PCR machine (Solas 8, MatMaCorp, Lincoln, Nebraska), which is a thermal cycler and also a real-time PCR machine. The real-time monitoring was done using EvaGreen (Biotium, San Francisco, California, USA) as the intercalating dye. The PCR primers used were for the human Cox-1 gene and the primers were Human cox1-F (Forward Primer) with the sequence identified as SEQ ID NO: 1 (5′-GGAGGAGACCCCATTCTATAC-3′) and the Human cox1-R (Reverse Primer) with the sequence identified as SEQ ID NO: 2 (5′-GCTCAGACCATACCTATGTATCC-3′). EvaGreen dye intercalation into the amplified products were monitored in real-time using the 520 nm Channel of the Solas 8 (MatMaCorp, Lincoln, Nebraska) device. A representative data from one of the samples is shown in FIG. 10, demonstrating that DNA is viable from the stored samples even after a month.

A system and method for collecting, storing, and analyzing a biological sample (nasal, oral, vaginal or anal specimens) using a swab without the use of any media to promote or enhance collection, transport, storage of at least fourteen days. The system includes the swab containing the collected biological sample, sealed within a flexible collection tube 200. The system may further include a vacuum bag that has been vacuum sealed to remove air, as well as moisture from the system, to promote long term storage (i.e. thirty days or more) of the stabilized biological sample prior to analysis.

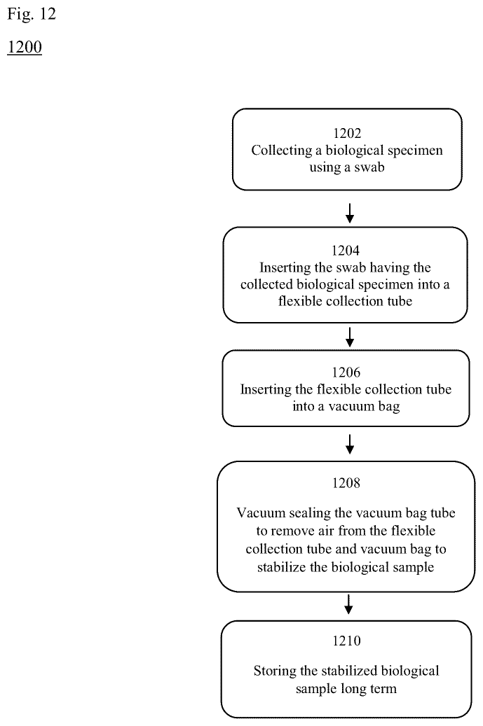

The method 1200, as represented in FIG. 12, for collection of a biological specimen using the system includes, obtaining the biological sample using the swab through conventional techniques (e.g. a cheek swab may be obtained according to the instructions in FIG. 13), inserting the swab into the flexible collection tube 200 and sealing the tube. The method may further include inserting the flexible collection tube 200 containing the swab into a vacuum bag. The method may further include vacuum sealing the vacuum bag to remove air and moisture from the vacuum bag and flexible collection tube 200 to stabilize the biological sample for long term storage.

A collection portion of the swab may be made of a material that is nylon, cotton, or other natural or synthetic fibrous material.

The flexible collection tube 200 of the system may be a conventional sample extraction tube that is made of a flexible material such as low-density polyethylene (LDPE).

FIG. 13 represents instructions for sample collection for use in the method 1200.

1. A biological specimen collection system for long term storage comprising: a swab for collecting the biological specimen, a flexible collection tube, and a sealed vacuum bag, the sealed vacuum bag comprising a vacuum bag that is vacuum sealed to remove air from the vacuum bag, and flexible collection tube, and where the system does not comprise any media for collection, transport, or storage of the biological specimen.
The system of claim 1, wherein the flexible collection tube comprises a main body made of a low-density polyethylene material.
The system of claim 2, wherein the flexible collection tube further comprises a cap that is in removable attachment to the main body, and a smaller cap in removable screw attachment to the cap.
A biological specimen collection system for storage of a biological sample comprising:
a swab for collecting the biological specimen, and a flexible collection tube, and where the system does not comprise any media for collection, transport, or storage of the biological specimen.
The system of claim 1, wherein the flexible collection tube comprises a main body made of a low-density polyethylene material.
The system of claim 2, wherein the flexible collection tube further comprises a cap that is in removable attachment to the main body, and a smaller cap in removable screw attachment to the cap.
A method for collection and storage of a biological specimen without the use of any media for collection, transport, or storage of the biological specimen, the method comprising:
collecting the biological specimen using a swab,
inserting the swab having the collected biological specimen into a flexible collection tube, and
inserting the flexible collection tube into a vacuum bag to stabilize the biological sample, and
storing the stabilized biological sample for at least 14 days.
The method of claim 7, wherein the flexible collection tube comprises a cap that is in removable attachment to the main body, and a smaller cap in removable screw attachment to the cap.
The method of claim 8 further comprising after the insertion of the flexible collection tube into the vacuum bag, vacuum sealing the vacuum bag to remove air from the flexible collection tube and vacuum bag to stabilize the biological sample, wherein the small cap of the flexible collection tube is partially unscrewed, and wherein the storing of the stabilized biological sample for long term storage of at least 30 days.
